(19) United States
(12) Patent Application Publication
(10) Pub. No.: US 2008/0090903 A1
(43) Pub. Date: Apr. 17, 2008

(54) PHENYLALKYL CARBAMATE COMPOSITIONS

(76) Inventors: Ramendra N. Pandey, Bridgewater, NJ (US); Tracey Mascaro, Hatfield, PA (US); Ronnie McDowell, Union, NJ (US); John Troisi, Neshanic Station, NJ (US); James McCool, Sellersville, PA (US); Stanley Altan, North Brunswick, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/868,588

(22) Filed: Oct. 8, 2007

Related U.S. Application Data
(60) Provisional application No. 60/829,355, filed on Oct. 13, 2006.

(51) Int. Cl. A61K 31/27; A61P 25/00

(52) U.S. Cl. 514/489

(57) ABSTRACT

The present invention relates to a composition of a phenylalkyl carbamate compound that results in improved stability, wherein the composition comprises a phenylalkyl carbamate compound in a mixture with an effective amount of one or more excipients and, wherein at least one excipient is dibasic calcium phosphate dihydrate.

### FIELD OF THE INVENTION

The invention is directed to a composition of a phenylalkyl carbamate compound that results in improved stability. More particularly, the compositions comprise a phenylalkyl carbamate compound in a mixture with dibasic calcium phosphate dihydrate that result in improved stability of the phenylalkyl carbamate compound.

### BACKGROUND OF THE INVENTION

The phenylalkyl carbamates described and included in the scope of this invention have been described in U.S. Pat. No. 3,265,728, U.S. Pat. No. 3,313,692, U.S. Pat. No. 6,103,759, U.S. Pat. No. 6,562,867, U.S. Pat. No. 6,541,513, U.S. Pat. No. 6,589,985 and U.S. Pat. No. 6,815,464 and PCT publications WO02/067924, WO02/067925, WO02/067924, WO02/067923, WO02/07822, WO03/007934 and WO03/007936, which are incorporated herein by reference in their entirety.

These compounds are pharmaceutically useful for treating and preventing central nervous system disorders including convulsions, epilepsy, stroke and muscle spasm; useful in the treatment of central nervous system diseases, particularly as anticonvulsants, antiepileptics, neuroprotective agents and centrally acting muscle relaxants; useful in treating and preventing neuropathic pain, cluster and migraine headache pain, bipolar disorder, chronic and acute neurodegenerative disorders, psychotic disorders, movement disorders, addictive disorders, impulse control disorders, anxiety disorders, antiepileptogenesis and for the treatment of pain.

Neuropathic pain is defined as pain caused by aberrant somatosensory processing in the peripheral or central nervous system and includes painful diabetic peripheral neuropathy, post-herpetic neuralgia, trigeminal neuralgia, post-stroke pain, multiple sclerosis-associated pain, neuropathies-associated pain such as in idiopathic or post-traumatic neuropathy and mononeuritis, HIV-associated neuropathic pain, cancer-associated neuropathic pain, carpal tunnel-associated neuropathic pain, spinal cord injury-associated pain, complex regional pain syndrome, fibromyalgia-associated neuropathic pain, lumbar and cervical pain, reflex sympathic dystrophy, phantom limb syndrome and other chronic and debilitating condition-associated pain syndromes.

Cluster headache (also called Raeder's syndrome, histamine cephalalgia and sphenopalatine neuralgia) is characterized by a series of short-lived attacks of periorbital pain on an almost daily basis over a relatively short period of time (for example, over 4 to 8 weeks) followed by a pain-free interval.

Migraine headache is also a periodic recurring disorder that can be associated with paroxysmal pain, vomiting, and photophobia. Migraine headaches include, and are not limited to, classic migraine (migraine with aura: associated with premonitory sensory, motor or visual symptoms) and common migraine (migraine without aura). Cluster and migraine headache-associated pain are also clinical indications with significant unmet medical need.

The described phenylalkyl carbamate compounds are susceptible to degradation above pH 5, which limits the shelf life of the compounds and compositions thereof. Therefore, there is a need to develop a robust composition of a phenylalkyl carbamate compound with improved stability of the compound. It is an object of the present invention to provide such a robust composition.

It has previously been disclosed that large particle sizes of dibasic calcium phosphate dihydrate (DCPD) when formulated as a tablet with aspirin has reduced the propensity of aspirin to degrade to salicylic acid and acetic acid compared to smaller particle sized DCPD (Landin et al., 1994, Int. J. Pharm. 107:247-249; Landin et al., 1995, Int. J. Pharm. 123:143-144). The mechanism for the degradation of aspirin to salicylic acid and acetic acid is hydrolysis (Leesen and Mattocks (1958) J. Am. Pharm. Sci. Ed., 67:329-333). The poorer stability of tablets containing powdered material of DCPD as compared to aggregated material was attributed to a greater propensity of smaller particle size DCPD to lose more water (Landin et al., 1994, 1995, supra).

U.S. Pat. No. 6,462,022 discloses the use of large particle sized DCPD (described as having a specific surface area of less than 1.5 m²g⁻¹ prior to compaction or tabletting) in a lisinopril formulation/composition to reduce the amount of the lisinopril degradation product DKP (diketopiperazine) that is formed, thereby increasing the shelf-life of tablets formulated with the larger sized DCPD, particularly those with low doses of lisinopril.

### SUMMARY OF THE INVENTION

The present invention is directed to a composition of a phenylalkyl carbamate compound comprising an admixture of the compound with an effective amount of one or more excipients wherein at least one excipient is dibasic calcium phosphate dihydrate, whereby the dibasic calcium phosphate dihydrate reduces degradation of the phenylalkyl carbamate compound in the composition.

Therefore, in one general aspect, the present invention provides a composition comprising an effective amount of one or more excipients wherein at least one excipient is dibasic calcium phosphate dihydrate and a compound of formula (I):

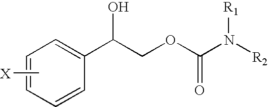

or a form thereof wherein
phenyl is substituted at X with one to five halogen atoms independently selected from the group consisting of fluorine, chlorine, bromine and iodine; and,
R₁ and R₂ are independently selected from the group consisting of hydrogen and C₁₋₄alkyl; wherein C₁₋₄alkyl is optionally substituted with phenyl, wherein phenyl is optionally substituted with substituents independently selected from the group consisting of halogen, C₁₋₄alkyl, C₁₋₄alkoxy, amino, nitro and cyano.

In an embodiment, the present invention provides a composition comprising an effective amount of one or more excipients wherein at least one excipient is dibasic calcium phosphate dihydrate and a carbamic acid 2-(2-chloro-phenyl)-2-hydroxy-ethyl ester compound of formula (Ia):

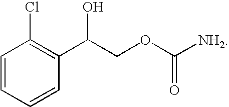

In another embodiment, the compositions of the present invention are tablets comprising an effective amount of dibasic calcium phosphate dihydrate and a carbamic acid 2-(2-chloro-phenyl)-2-hydroxy-ethyl ester compound of formula (Ia).

In another embodiment, the present invention provides a composition comprising an effective amount of one or more excipients wherein at least one excipient is dibasic calcium phosphate dihydrate and a carbamic acid (2R)-2-(2-chloro-phenyl)-2-hydroxy-ethyl ester compound of formula (Ib):

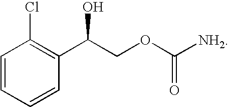

In another embodiment, the compositions of the present invention are tablets comprising an effective amount of dibasic calcium phosphate dihydrate and a carbamic acid (2R)-2-(2-chloro-phenyl)-2-hydroxy-ethyl ester compound of formula (Ib).

In another embodiment, carbamic acid (2R)-2-(2-chloro-phenyl)-2-hydroxy-ethyl ester compound of formula (Ib) predominates in a range of from about 75% or greater; or in a range of from about 90% or greater; or in a range of from about 95% or greater; or in a range of from about 98% or greater; or in a range of from about 99% or greater.

In another embodiment, the present invention provides a composition comprising an effective amount of one or more excipients wherein at least one excipient is dibasic calcium phosphate dihydrate and a carbamic acid (2S)-2-(2-chloro-phenyl)-2-hydroxy-ethyl ester compound of formula (Ic):

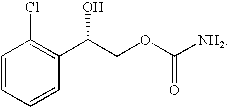

In another embodiment, the compositions of the present invention are tablets comprising an effective amount of dibasic calcium phosphate dihydrate and a carbamic acid (2S)-2-(2-chloro-phenyl)-2-hydroxy-ethyl ester compound of formula (Ic).

In another embodiment, carbamic acid (2S)-2-(2-chloro-phenyl)-2-hydroxy-ethyl ester compound of formula (Ic) predominates in a range of from about 75% or greater; or in a range of from about 90% or greater; or in a range of from about 95% or greater; or in a range of from about 98% or greater; or in a range of from about 99% or greater.

The present invention also provides methods of making and using the composition of the invention.

### DETAILED DESCRIPTION OF THE INVENTION

All publications cited herein are hereby incorporated by reference. Unless defined otherwise, all technical and scientific terms used herein have the same meaning as commonly understood to one of ordinary skill in the art to which this invention pertains.

The following abbreviations used in this specification have the following meanings: the term “API” means active pharmaceutical ingredient; “CNS” means central nervous system; “HPLC” means High Pressure Liquid Chromatography; and “RH” means Relative Humidity.

It must be noted that as used herein and in the appended claims, the singular forms “a,” “an,” and “the” include plural references unless the context clearly dictates otherwise. Thus, for example, a reference to “a phenylalkyl carbamate” is a reference to one or more phenylalkyl carbamates and includes equivalents thereof known to those skilled in the art and so forth.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, the terms “comprising”, “containing”, “having” and “including” are used in their open, non-limiting sense.

As used herein, the term “composition” is intended to encompass a product comprising the specified ingredients in the specified amounts, as well as any product which results, directly or indirectly, from combinations of the specified ingredients in the specified amounts. Furthermore, the term composition is used interchangebly with the term “formulation,” whereby both terms are intended to have a similar meaning and both of which, in addition to the foregoing definition, are intended to take on the ordinary meaning given to them by one skilled in the art.

As used here in, the term “dibasic calcium phosphate dihydrate” or “DCPD” is a chemical compound having the formula of CaHPO₄.2H₂O. Synonyms and trademarks for dibasic calcium phosphate dihydrate include: Cafos; calcium hydrogen orthophosphate dihydrate; calcium monohydrogen phosphate dihydrate; Calstar; Calipharm; dicalcium orthophosphate; Difos; DI-TAB; E341; Emcompress® (brand of DCPD); phosphoric acid calcium salt (1:1) dihydrate; secondary calcium phosphate; calcium phosphate; and dicalcium phosphate (DCP). The latter two terms are commonly used generic terms in the pharmaceutical art.

Dicalcium phosphate is believed to be alkaline (El-Shattaway, H H; Kildsig, D O; Peck, G E. Erythromycin direct compression excipients: preformulation stability screening using differential scanning calorimetry, Drug Dev. Ind. Pharm., 1982; 86: 937-947). In reality depending upon the degree of hydration, granulation, particle size (milled vs. unmilled), the surface pH of the dicalcium phosphate changes.

In this invention it is demonstrated that a dicalcium phosphate with a slightly acidic surface pH affords stability to the carbamic acid (2R)-2-(2-chloro-phenyl)-2-hydroxy-ethyl ester compound of formula (Ib). As surface pH increases, we have discovered that both hydrolysis and rearrangement degradation of the compound of formula (Ib) correspondingly increase.

As a result of this discovery, we now believe that for API's which are susceptible to physical and chemical degradation due to the surface pH of dicalcium phosphate or other excipients used in a formulation, irrespective of particle size, such degradation of the API may be reduced by using various forms of dicalcium phosphate either alone or in combination with such other excipients.

DCPD refers to commercially available grades of DCPD that are typically used in wet-granulated or roller-compacted formulations or in dry blend, direct-compression formulations. The milled grade of DCPD typically has a pH of about 6.5 to a pH of about 7. The unmilled grade of DCPD typically has an average pH of about 5.4.

DCPD is a white, odorless, tasteless, nonhygroscopic compound that is stable at room temperature. Under certain temperature and humidity conditions, DCPD loses water of crystallization below 100° C. Further, depending upon the degree of hydration, granulation (milled vs unmilled) and the like, the surface pH of the DCPD changes.

In the present invention, the use of commercially available unmilled DCPD is contemplated, wherein the unmilled DCPD has a pH in a range of from about 5.0 to a pH of about 5.8; or a pH in a range of from about 5.1 to a pH of about 5.7; or a pH in a range of from about 5.2 to a pH of about 5.6; or a pH in a range of from about 5.3 to a pH of about 5.5; or a pH in a range of about 5.4.

In the present invention, the use of unmilled DCPD having a pH in one or more of the foregoing pH ranges has the function of significantly reducing degradation of a phenylalkyl carbamate compound, thus resulting in improved stability of the compound. Such a function of unmilled DCPD is dependent on the structure of the compound and the presence of reactive groups.

DCPD can be used in both tablet and capsule formulations. DCPD may also be used both as an excipient and as a source of calcium in nutritional supplements. As a tablet excipient, DCPD is used because of its compaction properties and good-flow properties, particularly the unmilled material.

The term “tablet” means an API mixed with excipients and pressed into an oral dosage form.

A “capsule” is an oral dosage form in the shape of an oblong rounded container containing an API optionally mixed with excipients.

An “excipient” is generally an inactive substance used as a vehicle for an API. In addition, excipients can be used to aid the process by which a product is manufactured. An excipient is generally inactive, however, depending on the physical and chemical stability of the API, certain excipients can either degrade the API or can be used to stabilize the API. In a composition, using standard formulation techniques, the API may be dissolved or mixed with one or more optional excipients. The types of excipients used in a tablet include, but are not limited to, binders, fillers, disintegrants, lubricants, coatings, sweeteners, and flavors and colors. In many instances, one particular excipient may be used to perform more than one function, e.g., a binder may be used as a filler. In other instances, not every excipient is physically and chemically compatible with every API.

In addition, depending on the route of administration, taste of the drug or dosage form, various excipients may be used to enhance the pharmaceutical elegance of the composition.

A “binder” is generally an inactive ingredient used to hold the ingredients in a tablet together. A wide variety of binders can be used, including but not limited to, gum, wax, tapioca starch (cassava flour), polyethylene glycol, hydroxypropyl methylcellulose (HPMC), hydroxypropyl cellulose, and polyvinylpyrrolidone, etc. In some instances, a binder may be used as a filler.

A “filler” is generally an inactive substance used to fill out the size and shape of a tablet or capsule, making it practical to produce and convenient for the consumer to use, i.e., making a product bigger or easier to handle. Examples of fillers include, but are not limited to, cellulose, lactose, sucrose, mannitol, DCPD, microcrystalline cellulose (MCC), HPMC, soybean oil, safflower oil, ProSolv HD90 (brand of a co-processed mixture of MCC and colloidal silicon dioxide) and the like. In some instances, a binder may be used as a filler; for example, the binder cellulose or HPMC may be used as a filler in tablets or hard gelatin capsules. In another example, soybean or safflower oil is used as the filler in soft gelatin capsules.

A “disintegrant” is generally an inactive ingredient added to the tablet that readily absorbs water to help the tablet disperse once swallowed. A disintegrant expands when wet causing the tablet to break apart in the digestive tract, thus releasing the drug for absorption. Examples of disintegrants include, but are not limited to, sodium starch glycolate (SSG) and cross-linked polyplasdone (Crospovidone). Some binders, such as starch, are also used as disintegrants.

A “lubricant” is generally an inactive ingredient added to prevent other ingredients from clumping together and from sticking to equipment. Examples of lubricants include, but are not limited to, common minerals, talc, silica, stearic acid (stearin), magnesium stearate (MS), sodium lauryl sulfate (SLS), sodium stearyl fumarate (SSF) and colloidal silicon dioxide (CSD) and the like.

A “powder flow enhancer” or “glidant” is generally an inactive ingredient that functions as the name implies. Examples of lubricants that function as powder flow enhancers are CSD and talc.

The term “form” means, in reference to a compound of the present invention, that such may exist as, without limitation, a salt, stereoisomer, tautomer, crystalline, polymorph, amorphous, solvate, hydrate, ester, prodrug or metabolite form. The present invention encompasses all such compound forms and mixtures thereof.

The term “isolated form” means, in reference to a compound of the present invention, that such may exist in an essentially pure state such as, without limitation, an enantiomer, a racemic mixture, a geometric isomer (such as a cis or trans stereoisomer), a mixture of geometric isomers and the like. The present invention encompasses all such compound forms and mixtures thereof.

The compounds of the invention may be present in the form of pharmaceutically acceptable salts or esters. For use in medicines, the term “pharmaceutically acceptable salts or esters” shall mean non-toxic salts or esters of the compounds employed in this invention which are generally prepared by reacting the free acid with a suitable organic or inorganic base. Examples of such salts include, but are not limited to, acetate, benzenesulfonate, benzoate, bicarbonate, bisulfate, bitartrate, borate, bromide, calcium, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylbromide, methylnitrate, methylsulfate, mucate, napsylate, nitrate, oleate, oxalate, pamaote, palmitate, panthothenate, phosphate/diphosphate, polygalacturonate, potassium, salicylate, sodium, stearate, subacetate, succinate, tannate, tartrate, teoclate, tosylate, triethiodide, valerate and the like.

The invention includes compounds of various isomers and mixtures thereof. The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (optical isomers).

The term “optical isomer” means isomers of identical constitution that differ only in the spatial arrangement of their groups. Optical isomers rotate the plane of polarized light in different directions. The term “optical activity” means the degree to which an optical isomer rotates the plane of polarized light.

The term “racemate” or “racemic mixture” means an equimolar mixture of two enantiomeric species, wherein each isolated specie rotates the plane of polarized light in the opposite direction such that the mixture is devoid of optical activity.

The term “enantiomer” means an isomer having a nonsuperimposable mirror image. The term “diastereomer” means stereoisomers that are not enantiomers.

The term “chiral” means a molecule which, in a given configuration, cannot be superimposed on its mirror image. This is in contrast to achiral molecules which can be superimposed on their mirror images.

The two distinct mirror image versions of the chiral molecule are also known as levo (left-handed), abbreviated L, or dextro (right handed), abbreviated D, depending on which way they rotate polarized light. The symbols “R” and “S” represent the atom configuration of groups around a stereogenic carbon atom(s) and are intended to be used as defined in the literature.

An example of an enantiomerically enriched form isolated from a racemic mixture includes a dextrorotatory enantiomer, wherein the mixture is substantially free of the levorotatory isomer. In this context, substantially free means the levorotatory isomer may, in a range, comprise less than 25% of the mixture, less than 10%, less than 5%, less than 2% or less than 1% of the mixture according to the formula:

$$\% levoratory = \frac{( mass levoratory )}{( mass dextrototatory ) + ( mass levoratory )} \times 100$$

Similarly, an example of an enantiomerically enriched form isolated from a racemic mixture includes a levorotatory enantiomer, wherein the mixture is substantially free of the dextrorotatory isomer. In this context, substantially free means the dextrorotatory isomer may, in a range, comprise less than 25% of the mixture, less than 10%, less than 5%, less than 2% or less than 1% of the mixture according to the formula:

$$\% dextrototatory = \frac{( mass dextrototatory )}{( mass dextrototatory ) + ( mass levoratory )} \times 100$$

The compounds of the invention may be prepared as individual isomers by either isomer-specific synthesis or resolved from an isomeric mixture.

Furthermore, compounds of the present invention may have at least one crystalline, polymorph or amorphous form. The plurality of such forms are intended to be included in the scope of the invention. In addition, some of the compounds may form solvates with water (i.e., hydrates) or common organic solvents (e.g., organic esters such as ethanolate and the like). The plurality of such solvates are also intended to be encompassed within the scope of this invention.

The term “alkyl” means a saturated aliphatic branched or straight-chain hydrocarbon radical or linking group having from 1 up to 8 carbon atoms in a linear or branched arrangement. The term “alkyl” also includes a “lower alkyl” radical or linking group having from 1 up to 4 carbon atoms respectively, such as methyl, ethyl, 1-propyl, 2-propyl, 1-butyl, 2-butyl, tert-butyl, 1-pentyl, 2-pentyl, 3-pentyl, 1-hexyl, 2-hexyl, 3-hexyl, 1-heptyl, 2-heptyl, 3-heptyl, 1-octyl, 2-octyl, 3-octyl and the like. Alkyl radicals may be attached to a core molecule and further substituted on any atom when allowed by available valences.

The term “alkoxy” means an alkyl radical or linking group having from 1 up to 8 carbon atoms in a linear or branched arrangement, wherein the radical or linking group is attached through an oxygen linking atom, as in the formula: —O-alkyl. The term “alkoxy” also includes a “lower alkoxy” radical or linking group having from 1 up to 4 carbon atoms respectively, such as methoxy, ethoxy, propoxy, butoxy and the like. An alkoxy radical may be attached to a core molecule and further substituted on any carbon atom when allowed by available valences.

The term “halogen” means an atom selected from fluorine, chlorine, bromine and iodine.

A “tablet coating” protects tablet ingredients or tablet integrity from deterioration by moisture in the air and, in many cases, makes tablets easier to swallow. Some coatings are used to provide color or a smooth finish, or to facilitate printing on the tablet (although characters and symbols are easy to emboss into the tablets using special punches).

In one embodiment, a cellulose film coating is used which is free of sugar and potential allergy-causing substances. In another embodiment, other coating materials are used such as corn protein (zein) or an extraction from trees (pharmaceutical glaze).

Some tablets have a special coating termed an enteric coating, which is resistant to stomach acid and dissolves in the high pH of the intestines. The purpose of this coating is to prevent dissolution of the tablet in the stomach, where the stomach acid may degrade the active ingredient, or where the time of passage may compromise its effectiveness, in favor of dissolution in the small intestine, where the active principle is better absorbed.

A “release coating” controls the rate of drug release, or controls specifically when the drug will be released in the digestive tract. Coating is also used for product identification and differentiation.

As used herein, “ambient conditions” are the conditions measured in the immediate area surrounding a composition of the invention. This term can be applied to any unit of measure, such as temperature, pressure, humidity, light intensity, etc. For example, ambient conditions can be used to refer to a combination of a given temperature and relative humidity, such as 25° C. and 20% RH.

Under certain conditions of elevated temperature and relative humidity, such as, 25° C. and 40% RH, 25° C. and 60% RH, 25° C. and 80% RH, 45° C. and 20% RH, 45° C. and 40% RH, 45° C. and 60% RH, 45° C. and 80% RH, or 40° C. and75% RH and the like, an exposed compound or composition may be subject to degradation. Forced degradation studies showed that a compound of formula (I) was more susceptible to carbamate rearrangement and hydrolysis at a higher pH (pH 5 and above), the rate of which increased as pH increased.

In this invention, it has been discovered that unmilled DCPD provides protection against such hydrolysis and rearrangement degradation.

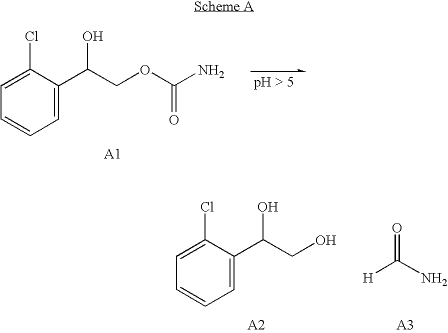

For a carbamic acid 2-(2-chloro-phenyl)-2-hydroxy-ethyl ester compound of formula (Ib), shown here as Compound A1, a formulation pH greater than pH 5 irreversibly shifts the equilibrium to cleave the carbamate group from Compound A1 to provide a mixture of hydrolysis products: 1-(2-chloro-phenyl)-ethane-1,2-diol Compound A2 and formamide Compound A3.

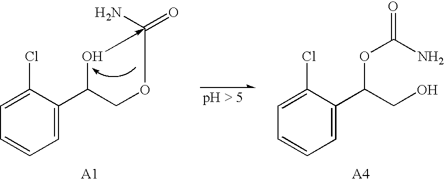

Compound A1 is also subject to in situ rearrangement at a formulation pH greater than pH 5, which results in carbamic acid 1-(2-chloro-phenyl)-2-hydroxy-ethyl ester Compound A4.

It will be appreciated that there will be potential improvements in shelf-life of compounds of formula (I) in a composition containing unmilled DCPD. Therefore, in one general aspect, the present invention provides a composition comprising an effective amount of unmilled dibasic calcium phosphate dihydrate and a compound of formula (I).

As used herein, an “effective amount of dibasic calcium phosphate dihydrate” means that amount of DCPD added to a composition that makes a compound of formula (I) stable in the composition. For example, an “effective amount of dibasic calcium phosphate dihydrate” can be the amount of DCPD added to a composition that decreases the physical or chemical degradation of a compound of formula (I) in the composition. It is readily appreciated that the effective amount of DCPD can vary depending upon the particular compound of formula (I), the dose range of the compound and the presence of other excipients in the composition, etc. Methods are known in the art for determining the “effective amount of DCPD”. For example, a skilled artisan can determine the effective amount of DCPD experimentally by making blends containing a compound of formula (I), DCPD and other excipients, subjecting the blends to elevated temperature and relative humidity storage for accelerated degradation, and measuring the amount of compound degradation.

The “effective amount of DCPD” is about 4% (w/w) of the composition to obtain the benefit of the invention. Furthermore, embodiments intended to be included within the scope of the present include an “effective amount of DCPD” of about 4% (w/w), 6% (w/w), 8% (w/w), 10% (w/w), 12% (w/w), 14% (w/w), 16% (w/w), 18% (w/w), 20% (w/w), 22% (w/w), 24% (w/w), 26% (w/w), 28% (w/w), 30% (w/w), 32% (w/w), 34% (w/w), 36% (w/w), 38% (w/w), 40% (w/w), 42% (w/w), 44% (w/w), 46% (w/w), 48% (w/w), 50% (w/w), 60% (w/w), 70% (w/w), and the like of the composition.

Embodiments of the present invention include an effective amount of DCPD in a range of from about 4% (w/w) to about 40% (w/w), a range of from about 4% (w/w) to about 35% (w/w), a range of from about 4% (w/w) to about 30% (w/w), a range of from about 4% (w/w) to about 25% (w/w), a range of from about 4% (w/w) to about 20% (w/w), a range of from about 4% (w/w) to about 10% (w/w) and a range of about 4%.

The term “stable” as used herein, refers to the tendency of a compound or a composition to remain substantially in the same physical and chemical form for a period of 6 months; or, a period of one year; or, a period of two years; or, a period of 3 years; or, a period of 4 years; or, a period of 5 years, when stored under ambient conditions.

Embodiments of the present invention include compositions that remain stable for a period of time in a range of about 6 months to about 5 years; or, in a range of from about one year to about 5 years; or, in a range of from about 2 years to about 5 years; or, in a range of from about 3 years to about 5 years; or, in a range of from about 4 years to about 5 years; or, in a range of about 5 years, when stored under ambient conditions.

In another embodiment, the present invention provides a tablet comprising a compound of formula (I) and an effective amount of DCPD. The invention is not limited by the tabletting method. The tablets of the present invention can be formed by either the wet-granulated method or by a dry blend, direct-compression tabletting method.

In still another embodiment, the present invention provides a tablet comprising a compound of formula (I) and an effective amount of commercially available unmilled DCPD prepared in a dry granulation and a direct compression tabletting method.

The composition of the present invention can optionally further comprise additional diluents or excipients and other therapeutic agents.

Embodiments of the present invention include a composition further comprising an additional excipient selected from microcrystalline cellulose, hydroxypropyl methylcellulose, lactose, mannitol, sodium starch glycolate, cross-linked polyplasdone, polyethylene glycol, sodium lauryl sulfate, magnesium stearate, sodium stearyl fumarate or colloidal silicon dioxide

An embodiment of the present invention includes a composition comprising an additional excipient selected from hydroxypropyl methylcellulose, sodium starch glycolate, cross-linked polyplasdone, polyethylene glycol, sodium lauryl sulfate or colloidal silicon dioxide.

An embodiment of the present invention includes a composition comprising an additional excipient selected from hydroxypropyl methylcellulose or sodium starch glycolate.

For example, a composition of the present invention can comprise a carbamic acid (2R)-2-(2-chloro-phenyl)-2-hydroxy-ethyl ester compound of formula (Ib) as the API, DCPD, HPMC or PEG and SSG or Crospovidone. The tablet can further optionally comprise one or more of SLS or CSD.

Another embodiment of the present invention includes a composition comprising one or more of an excipient selected from HPMC or Crospovidone.

The present invention also provides a method of preparing the composition of the invention comprising the step of admixing an effective amount of one or more excipients wherein at least one excipient is DCPD with a compound of formula (I). The compositions may be conveniently presented in unit dosage forms, and prepared by any methods known in the art of pharmacy.

To prepare the pharmaceutical compositions of this invention, one or more compounds of formula (I) or salt thereof as the active ingredient is intimately admixed with an effective amount of DCPD and a pharmaceutically acceptable carrier according to conventional pharmaceutical compounding techniques. Carriers are generally necessary and inert pharmaceutical excipients, including, but not limited to, binders, fillers, disintegrants, suspending agents, lubricants, flavorings, sweeteners, preservatives, dyes and coatings. In preparing compositions in oral dosage form, any of the usual pharmaceutical carriers may be employed which provide a stable dosage form. For example, for solid oral preparations, suitable carriers and additives include starches, sugars, diluents, granulating agents, lubricants, binders, disintegrating agents and the like.

Any solid form of a compound of formula (I) can be used in the invention including, but not limited to, a salt, stereoisomer (such as an enantiomer or a racemic mixture), tautomer, crystalline, polymorph, amorphous, solvate, hydrate, ester, prodrug or metabolite form. The present invention encompasses all such compound forms and mixtures thereof.

Commercially available grades of unmilled DCPD are commonly used in direct compression/compaction or dry granulation techniques and are used in the present invention.

The compounds of formula (I) can be synthesized by methods known to those skilled in the art, as described in U.S. Pat. No. 3,265,728, U.S. Pat. No. 3,313,692, U.S. Pat. No. 6,103,759, U.S. Pat. No. 6,562,867, U.S. Pat. No. 6,541,513, U.S. Pat. No. 6,589,985 and U.S. Pat. No. 6,815,464 and PCT publications WO02/067924, WO02/067925, WO02/067924, WO02/067923, WO02/07822, WO03/007934 and WO03/007936, which are incorporated herein by reference in their entirety.

The salts and esters of the compounds of formula (I) can be produced by treating the compound with an acid in suitable solvent or by means well known to those of skill in the art.

The invention also provides the use of a composition of the invention, for example, in the treatment of CNS disorders. The term “CNS disorders” means a disorder selected from CNS disorders, such as pain, depression, anxiety, epilepsy, stroke, dementia and Parkinson's disease.

The invention further provides the use of an effective amount of DCPD and a compound of formula (I) in the manufacture of a medicament for the treatment of CNS disorders.

The present invention further provides a method for the treatment of CNS disorders in a subject in need thereof comprising administering to the subject a therapeutically or prophylactically effective amount of a composition comprising an effective amount of dibasic calcium phosphate dihydrate and a compound of formula (I). The method also comprises administering to the subject a prophylactically effective amount of a composition comprising an effective amount of dibasic calcium phosphate dihydrate and a compound of formula (I).

The terms “subject” and “patient” are used herein interchangeably and as used herein refer to an animal, preferably a mammal, and most preferably a human, who has been the object of treatment, observation or experiment. The term mammals include human patients and non-human primates, as well as experimental animals such as rabbits, rats, mice and other like animals.

Therefore, the term “a subject in need of treatment” as used herein will refer to a subject or patient who currently has or may develop a CNS disorder, including any mood disorder which can be treated by a therapeutic agent, or any other disorder in which the patient's present clinical condition or prognosis could benefit from the administration of one or more compounds of formula (I) alone or in combination with another therapeutic intervention including but not limited to another therapeutic agent.

The term “therapeutically effective amount” as used herein means a sufficient amount of one or more of the compounds of the invention to produce a therapeutic effect, as defined above, in a subject or patient in need of such treatment.

The term “prophylactically effective amount” is intended to mean that amount of a pharmaceutical drug that will prevent or reduce the risk of occurrence of the biological or medical event that is sought to be prevented in a tissue or a system, animal or human that is being sought by a researcher, veterinarian, medical doctor or other clinician.

Methods are known in the art for determining therapeutically and prophylactically effective doses for the instant pharmaceutical composition. For example, for use as an adjunct for treating CNS disorders, the compound can be employed at a daily dose in the range of about 0.1 mg to 400 mg usually in a regimen of 1 to 2 times per day, for an average adult human. The effective amount, however, may be varied depending upon the particular compound used, the mode of administration, the strength of the preparation and the advancement of the disease condition. In addition, factors associated with the particular patient being treated, including patient age, weight, diet, time of administration and response to treatment, will result in the need to adjust dosages.

Because of their ease in administration, tablets and capsules represent the most advantageous oral dosage unit form for the composition of the present invention. If desired, tablets may be sugar coated or enteric coated by standard techniques. The tablets or capsules can be coated or otherwise compounded to provide a dosage form affording the advantage of prolonged action. For example, the tablet or pills can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The composition of the present invention may be used in a unit dosage form such as a tablet, capsule, powder or granule.

The pharmaceutical compositions herein will contain, per dosage unit, e.g., tablet, capsule or powder, an amount of the active ingredient necessary to deliver a therapeutically or prophylactically effective dose as described above. For example, the pharmaceutical compositions herein can contain, per unit dosage unit, a therapeutically or prophylactically effective dose in a range of from about 25 to about 400 mg of the active ingredient, or a dose in a range of from about 50 to about 200 mg of the active ingredient.

In some embodiments of the present invention, compositions of this invention may be administered as a combination product either singly or concomitantly with one or more other compound or therapeutic agent, e.g., with other antidepressant agents. In these embodiments, the present invention provides methods to treat or prevent CNS disorders in a patient. The method includes the step of; administering to the patient in need of treatment a therapeutically or prophylactically effective amount of one of the compounds of formula (I) disclosed herein in combination with an effective amount of one or more other compounds or therapeutic agents that have the ability to augment or synergistically augment the therapeutic effects of the compounds of the present invention.

“Concomitant administration” or “combination administration” of a compound, therapeutic agent or known drug with a composition of the present invention means administration of one or more other therapeutic agents and, in addition, the one or more compositions of the invention at such time that both the other therapeutic agents and the compound of formula (I) will have a therapeutic effect. In some cases this therapeutic effect will be synergistic. Such concomitant administration can involve concurrent (i.e. at the same time), prior, or subsequent administration of the therapeutic agent with respect to the administration of a compound of the present invention. A person of ordinary skill in the art would have no difficulty determining the appropriate timing, sequence and dosages of administration for particular therapeutic agents and compounds of the present invention.

In addition, in some embodiments, the composition of the present invention may be used, either alone or in combination with one or more other therapeutic agents as described above, or their salts or esters, for manufacturing a medicament for the purpose of providing adjuvant treatment to a patient or subject in need thereof.

### EXAMPLES

This invention will be better understood by reference to the examples that follow. Those skilled in the art will readily appreciate that these examples are only illustrative of the invention. Accordingly, the pharmaceutical composition of the present invention should not be construed as being limited by the methods and conditions expressed for preparing the various blends herein. One skilled in the art would know how to prepare the instant pharmaceutical composition through routine variations in such methods and conditions. The pharmaceutical composition of the present invention is described more fully in the claims that follow hereafter.

### Example 1

### Degradation Products

As shown in Scheme A, a sample of Compound A1, the carbamic acid (2R)-2-(2-chloro-phenyl)-2-hydroxy-ethyl ester compound of formula (Ib), was dissolved in a pH 9 carbonate buffer. The solution was allowed to stand at room temperature until the 1-(2-chloro-phenyl)-ethane-1,2-diol Compound A3, formamide Compound A4 and carbamic acid 1-(2-chloro-phenyl)-2-hydroxy-ethyl ester Compound A5 degradation products had formed in appreciable amounts.

The sample was analyzed by LC-MS under reverse phase conditions using positive ion ESI and APCI detection. Compound A1 exhibited a [M+H]⁺ peak of m/z 216. The major rearrangement degradation product Compound A5 also displayed a [M+H]⁺ peak of m/z 216, indicating that it is an isomer of Compound A1. The minor hydrolysis degradation product Compound A3 showed no signal, since Compound A3 does not have any strongly basic sites and therefore would have the potential not to produce a signal in a positive ion ESI or APCI experiment.

### Example 2

### Degradation Studies

The degradation study was run in two different phases, the Mass Blends and the N-1 Design. The Mass Blend study was designed to prove that all proposed excipient options mixed with the Active Pharmaceutical Ingredient (API) of the present invention would not show degradation products when placed on stability under stressed conditions. The excipient options included different classes of excipients that were needed such as Fillers, Binders, Disintegrants, Flow agents, Wetting agents, and Lubricants. The proposed excipients included DCPD as a solid dose filler. DCPD was a concern because of its alkalinity.

Accordingly, the DCPD was separated into a specific Mass Blend to show that the use of this excipient had a negative effect on total degradants. Surprisingly, the mass blend with DCPD resulted in lower degradation products than the mass blend without DCPD.

Since the results were contrary to expectation, a second excipient compatibility study was performed using an N-1 Statistical Design. The N-1 Design Study is able to distinguish the positive or negative contribution of each excipient and shows interactions that exist in a blend containing multiple excipients and an API compared to studies of traditional binary mixtures of API and individual excipients.

The N-1 Design demonstrated that blends with DCPD provided lower degradation than the other filler options in the study. The data was analyzed by using Mini Tab (a statistical program) and the data suggests that DCPD affords protection against degradation of the API of the present invention by lowering blend pH.

The studies performed are provided as follows:

### Mass Blend and N-1 Design Study Formulations:

TABLE 1
Mass Blend - 11 Excipient Placebo Formula

| Ingredient | Amt/Batch (g) | % w/w |
|---|---|---|
| Microcrystalline Cellulose - (Avicel PH101) | 50.000 | 20.0 |
| Sodium Starch Glycolate - (Primojel) | 20.000 | 8.0 |
| Magnesium Stearate - (Non-Bovine) | 5.000 | 2.0 |
| Lactose - (Anhydrous - Direct Tabletting) | 44.375 | 17.8 |
| Dibasic Calcium Phosphate Dihydrate- | 44.375 | 17.8 |
| (Emcompress) | | |
| Sodium Lauryl Sulfate | 5.000 | 2.0 |
| Polysorbate 80 | 5.000 | 2.0 |
| HPMC (5 cps) | 25.000 | 10.0 |
| Polyethylene Glycol - (Lutrol E1450) | 25.000 | 10.0 |
| Colloidal Silicone Dioxide - (Cab-O-Sil) | 1.250 | 0.5 |
| Gelucire 44/14 | 25.000 | 10.0 |

TABLE 2
Mass Blend - 11 Excipient Active Formula

| Ingredient | Amt/Batch (g) | % w/w |
|---|---|---|
| carbamic acid (2R)-2-(2-chloro-phenyl)-2- | 71.125 | 28.5 |
| hydroxy-ethyl ester compound of formula (lb) | | |
| Microcrystalline Cellulose - (Avicel PH101) | 25.000 | 10.0 |
| Sodium Starch Glycolate - (Primojel) | 12.500 | 5.0 |
| Magnesium Stearate - (Non-Bovine) | 5.000 | 2.0 |
| Lactose - (Anhydrous - Direct Tabletting) | 25.000 | 10.0 |
| Dibasic Calcium Phosphate Dihydrate- | 25.000 | 10.0 |
| (Emcompress) | | |
| Sodium Lauryl Sulfate | 5.000 | 2.0 |
| Polysorbate 80 | 5.000 | 2.0 |
| HPMC (5 cps) | 25.000 | 10.0 |
| Polyethylene Glycol - (Lutrol E1450) | 25.000 | 10.0 |
| Colloidal Silicone Dioxide - (Cab-O-Sil) | 1.250 | 0.5 |
| Gelucire 44/14 | 25.000 | 10.0 |

TABLE 3
Mass Blend - 10 Excipient Active Formula

| | Amt/ | |
|---|---|---|
| Ingredient | Batch (g) | % w/w |
| carbamic acid (2R)-2-(2-chloro-phenyl)-2-hydroxy- | 96.250 | 38.5 |
| ethyl ester compound of formula (lb) | | |
| Microcrystalline Cellulose - (Avicel PH101) | 25.000 | 10.0 |
| Sodium Starch Glycolate - (Primojel) | 12.500 | 5.0 |
| Magnesium Stearate - (Non-Bovine) | 5.000 | 2.0 |
| Lactose - (Anhydrous - Direct Tabletting) | 25.000 | 10.0 |
| Sodium Lauryl Sulfate | 5.000 | 2.0 |
| Polysorbate 80 | 5.000 | 2.0 |
| HPMC (5 cps) | 25.000 | 10.0 |
| Polyethylene Glycol - (Lutrol E1450) | 25.000 | 10.0 |
| Colloidal Silicone Dioxide - (Cab-O-Sil) | 1.250 | 0.5 |
| Gelucire 44/14 | 25.000 | 10.0 |

TABLE 4
N-1 Design Formula

| Ingredient | % w/w |
|---|---|
| carbamic acid (2R)-2-(2-chloro-phenyl)-2-hydroxy-ethyl | 50.0 |
| ester compound of formula (lb) | |
| Microcrystalline Cellulose - (Avicel PH101) | 17.5 |
| Magnesium Stearate - (Non-Bovine) | 2.0 |
| Lactose - (Anhydrous - Direct Tabletting) | 10.0 |
| Dibasic Calcium Phosphate Dihydrate-(Emcompress) | 10.0 |
| Sodium Starch Glycolate - (Primojel) | 8.0 |
| Crospovidone (Polyplasdone XL-10) | 8.0 |
| HPMC (Methocel 5 cps) | 10.0 |
| Polyethylene Glycol - (Lutrol E1450) | 10.0 |
| Colloidal Silicone Dioxide - (Cab-O-Sil) | 0.5 |
| Sodium Lauryl Sulfate (Empicol) | 2.0 |

### Blending

Two different blending processes were used between the Mass Blend study and the N-1 Design study. The mass blends included some liquid materials (polysorbate 80 and Gelucire 44/14). Since Gelucire is a waxy material at room temperature, the material needed to be melted into a liquid state by heating to 60° C., thus assuring that the material could be properly mixed. The following two sections detail the blending processes.

### Mass Blend Preparation:

1. Blend all previously weighed ingredients in a mortar and pestle using the order of addition defined in the following steps.
2. Add all dry ingredients in the following order with blending after each addition: carbamic acid (2R)-2-(2-chloro-phenyl)-2-hydroxy-ethyl ester compound of formula (Ib), microcrystalline cellulose, sodium starch glycolate, magnesium stearate, lactose, dibasic calcium phosphate dihydrate and sodium lauryl sulfate. Blend dry ingredients.
3. Add polysorbate 80 and continue blending.
4. Add previously heated and melted Gelucire 44/14 continue blending.

### N-1 Design Blend Preparation

1. Weigh ingredients: carbamic acid (2R)-2-(2-chloro-phenyl)-2-hydroxy-ethyl ester compound of formula (Ib), filler, disintegrant, binder, wetting agent, flow agent and lubricant. Blend all ingredients in a mortar and pestle using the following steps:
2. Add all ingredients to the mortar and pestle in the following order with blending after each addition: carbamic acid (2R)-2-(2-chloro-phenyl)-2-hydroxy-ethyl ester compound of formula (Ib), filler, disintegrant, binder, wetting agent, flow agent, and lubricant.
3. Fill blends into 160 mm×10 mm glass tubes.

### N-1 Design Study

Determination of possible incompatibilities between an API and different excipients is an important aspect of development of a solid oral dosage form. In order to develop a robust composition, an excipient compatibility study is designed and performed.

The general design of an excipient compatibility study involves an experiment where a systematic selection of all possible combinations of excipients selected for a particular API are tested. Each composition blend comprises the excipients selected but omits one excipient until all combinations of selected excipients have been tested. according to the formula:

$${\sum }_{j = 1}^{k} {\prod }_{{i = 1}_{i \neq j}}^{k} {l}_{i}$$

where k defines the number of excipient classes and each excipient class has a level lⱼ, where the level j is the series: 1,2, . . . , k. In this case, the sum k is 4, where the selection of excipients corresponds to filler, disintegrant, lubricant and flow enhancer.

The typical composition of a tablet formulation consists of the API and excipients, such as a binder, a filler, a disintegrant and a powder flow enhancer or a lubricant. It is appreciated that experimental methods used herein are readily applicable to compositions comprising different APIs and different excipients.

All excipients tested were obtained from commercial sources: DCPD (JRS Pharma, Patterson, N.Y.); lactose (Foremost, Rothschild, Wis.); MCC (FMC Bioploymer, Philadelphia, Pa.); Crospovidone (ISP Technologies, Kalvert City, Ky.); sodium starch glycolate (JRS Pharma, Patterson, N.Y.); magnesium stearate (Mallinckrodt, St. Louis, Mo.); colloidal silicon dioxide (Cabot, Tuscola, Ill.); sodium lauryl sulfate (Mutchler Inc. Cayey, PR.); polysorbate 80 (EM Science, Gibbstown, N.J.); Gelucire 44/14 (Gattefosse, Westwood, N.J.); HPMC (The Dow Chemical Company, Midland, Mich.); and, polyethylene glycol (BASF, Florham Park, N.J.).

### N-1 Design Blends

The excipient compatibility study consisted of 30 composition blends. One composition blend contained only the active ingredients and one other blend contained only the API. These two composition blends were used as controls. The blend compositions were based on a tablet dose of 250 mg.

TABLE 5
N-1 Design Blend Nomenclature

| Key | Function/Class | Ingredient |
|---|---|---|
| F1 | Filler | Lactose - (Anhydrous - Direct Tabletting) |
| F2 | Filler | Dibasic Calcium Phosphate Dihydrate- |
| | | (Emcompress) |
| D1 | Disintegrant | Sodium Starch Glycolate - (Primojel) |
| D2 | Disintegrant | Crospovidone (Polyplasdone XL-10) |
| W | Wetting agent | Sodium Lauryl Sulfate (Empicol) |
| B1 | Binder | HPMC (Methocel 5 cps) |
| B2 | Binder | Polyethylene Glycol - (Lutrol E1450) |
| Fa | Flow Agent | Colloidal Silicone Dioxide - (Cab-O-Sil) |
| A1 | Active Blend | carbamic acid (2R)-2-(2-chloro-phenyl)-2- |
| | | hydroxy-ethyl ester compound of formula (lb) |
| A2 | Active Blend | Microcrystalline Cellulose - (Avicel PH101) |
| A3 | Active Blend | Magnesium Stearate - (Non-Bovine) |

TABLE 6
N-1 Design Blend Compositions

| Blend | Dose (mg) | F1 | F2 | D1 | D2 | W | B1 | B2 | Fa | A |
|---|---|---|---|---|---|---|---|---|---|---|
| 1 | 450 | 0 | 0 | 1 | 0 | 1 | 1 | 0 | 1 | 1 |
| 2 | 450 | 0 | 0 | 1 | 0 | 1 | 0 | 1 | 1 | 1 |
| 3 | 450 | 0 | 0 | 0 | 1 | 1 | 1 | 0 | 1 | 1 |
| 4 | 450 | 0 | 0 | 0 | 1 | 1 | 0 | 1 | 1 | 1 |
| 5 | 460 | 1 | 0 | 0 | 0 | 1 | 1 | 0 | 1 | 1 |
| 6 | 460 | 1 | 0 | 0 | 0 | 1 | 0 | 1 | 1 | 1 |
| 7 | 490 | 1 | 0 | 1 | 0 | 0 | 1 | 0 | 1 | 1 |
| 8 | 490 | 1 | 0 | 1 | 0 | 0 | 0 | 1 | 1 | 1 |
| 9 | 450 | 1 | 0 | 1 | 0 | 1 | 0 | 0 | 1 | 1 |
| 10 | 497.5 | 1 | 0 | 1 | 0 | 1 | 1 | 0 | 0 | 1 |
| 11 | 497.5 | 1 | 0 | 1 | 0 | 1 | 0 | 1 | 0 | 1 |
| 12 | 490 | 1 | 0 | 0 | 1 | 0 | 1 | 0 | 1 | 1 |
| 13 | 490 | 1 | 0 | 0 | 1 | 0 | 0 | 1 | 1 | 1 |
| 14 | 450 | 1 | 0 | 0 | 1 | 1 | 0 | 0 | 1 | 1 |
| 15 | 497.5 | 1 | 0 | 0 | 1 | 1 | 1 | 0 | 0 | 1 |
| 16 | 497.5 | 1 | 0 | 0 | 1 | 1 | 0 | 1 | 0 | 1 |
| 17 | 460 | 0 | 1 | 0 | 0 | 1 | 1 | 0 | 1 | 1 |
| 18 | 460 | 0 | 1 | 0 | 0 | 1 | 0 | 1 | 1 | 1 |
| 19 | 490 | 0 | 1 | 1 | 0 | 0 | 1 | 0 | 1 | 1 |
| 20 | 490 | 0 | 1 | 1 | 0 | 0 | 0 | 1 | 1 | 1 |
| 21 | 450 | 0 | 1 | 1 | 0 | 1 | 0 | 0 | 1 | 1 |
| 22 | 497.5 | 0 | 1 | 1 | 0 | 1 | 1 | 0 | 0 | 1 |
| 23 | 497.5 | 0 | 1 | 1 | 0 | 1 | 0 | 1 | 0 | 1 |
| 24 | 490 | 0 | 1 | 0 | 1 | 0 | 1 | 0 | 1 | 1 |
| 25 | 490 | 0 | 1 | 0 | 1 | 0 | 0 | 1 | 1 | 1 |
| 26 | 450 | 0 | 1 | 0 | 1 | 1 | 0 | 0 | 1 | 1 |
| 27 | 497.5 | 0 | 1 | 0 | 1 | 1 | 1 | 0 | 0 | 1 |
| 28 | 497.5 | 0 | 1 | 0 | 1 | 1 | 0 | 1 | 0 | 1 |
| 29 | 347.5 | 0 | 0 | 0 | 0 | 0 | 0 | 0 | 0 | 1 |
| 30 | 250 | 0 | 0 | 0 | 0 | 0 | 0 | 0 | 0 | 0 |

0 = excipient not present in the blend
1 = excipient present in the blend

### Stability Protocol

### Preparation Of Stability Samples

Bulk blends were dispensed (approximately 88-92 mg) into 160 mm×10 mm glass tubes. Following weighing, each set of 34 blends were placed in a plastic test tube racks. Open test tube tops in each rack were covered with a large piece of a single layer of thin paper towel for easy equilibration of humidity inside the test tubes. 6 test racks were placed at 60° C. 75% RH , 6 at 40° C. 75% RH, 4 at 25° C. 60% RH, and 2 at 4° C.

At predetermined time points, samples were pulled out of the specific chambers, allowed to equilibrate at room temperature for 2 hrs, visually inspected and analyzed by HPLC for chemicall degradation. The testing performed at time zero and each interval was Appearance, Weight gain or loss, Assay & Impurities, and Control.

### Mass Blend Protocol

The three mass blend samples (11 Excipient Placebo, 11 Excipient Active, 10 Excipient Active and N-1 Design) were placed on 24 month stability. Each approximately 10 g sample was stored in open 1 oz. amber glass vials with an air-permeable permeable dust cover. The storage conditions and testing intervals are provided in Table 7.

TABLE 7
Storage conditions for Mass Blends

| Storage | 1 Month | 3 Month |
|---|---|---|
| 5° C. | C | C |
| 25° C./60% | X | X |
| 40° C./75% | X | X |
| 60° C./80% | X | |

### N-1 Design Protocol

The 30 sample blends from Table 6 were placed on 24 month stability. Each approximately 88-92 mg sample was stored in open 160 mm×10 mm glass tubes with an air-permeable dust cover. The storage conditions and testing intervals are provided in Table 8.

TABLE 8
Storage conditions for N-1 blends

| | | | | 3 |
|---|---|---|---|---|
| Storage | 14 day | 1 Month | 2 Month | Month |
| 4° C. | | C | | C |
| 25° C./60% | | | | |
| 40° C./75% | | X | X | X |
| 60° C./75% | X | X | | |

### Example3

### Stability Study Results

### Physical Appearance

Tubes containing blends were visually inspected, individually, against a bright light and the color of the blends were recorded. Visual appearance of the blends are recorded in Tables 1-A to 1-D.

### HPLC Analysis

HPLC System: Agilent 1100 HPLC System (or equivalent) with UV detection of 211 nm and 25 μL injection volume
HPLC Column: Waters Symmetry C18, 4.6×250 mm, 5 μm (or equivalent)
Column Temperature: 30° C.
Flow Rate: 1.5 mL/ minute
Detection: 211-nm
Run Time: 20 minutes
Injection Volume: 25 μL
Rinse Solvent: Water/Acetonitrile, 82/18 (v/v)
Mobile Phase: 0.170 M Sodium Phosphate Buffer/Acetonitrile, 82/18 (v/v) Buffer pH 3.0
Retention Time: Approximately 12.6 minutes
Acetonitrile: HPLC Grade
Water: 18 Mohm (minimum) Milli-Q® Water
Methanol: HPLC Grade
o-Phosphoric Acid, 85% ACS Grade
Potassium Dihydrogen Phosphate N.F. Food Grade (KH₂PO₄, anhydrous)

### System Suitability

1. The retention time of the RWJ-333369-000 peak is approximately 12.6 minutes.
2. The signal-to-noise ratio of the RWJ-333369-000 peak in the Sensitivity Solution must be 10 or larger.
3. The tailing factor for RWJ-333369-000 as calculated by the current USP method must be less than 2.0.

### Statistical Analysis

The statistical analysis of the study results was carried out through a series of non-indepenent ANOVAs, each ANOVA corresponding to a subset of runs with each subset characterized by the removal of 1 excipient class. For example, if the level lj is k, then there were k excipient classes. In this case, there were four excipient classes, resulting in four ANOVAs carried out. The error term was estimated from the residual error. Graphical methods were used to enable scientific interpretation of the results.

### Visual Appearance

For visual appearance testing, a small portion of the blend to be tested was removed from the stability bottle and placed on a white paper individually. Visual appearance was recorded in Table 9. ND means that visual appearance for the the blend stored under the indicated storage conditions was not determined.

TABLE 9
Mass Blend Appearance - 1 Month Data

| Mass Blends | Storage | Appearance |
|---|---|---|
| 11 Excipient | 2-8° C. | White Powder |
| Placebo | 25° C./60% RH | Cracked white powder |
| | 40° C./75% RH | Cracked white to off white powder |
| | 60° C./80% RH | Cracked light yellow powder |
| 11 Excipient | 2-8° C. | ND |
| Active | 25° C./60% RH | ND |
| | 40° C./75% RH | Cracked white to off white powder |
| | 60° C./80% RH | Cracked light yellow powder |
| 10 Excipient | 2-8° C. | White powder |
| Active | 25° C./60% RH | Cracked white powder |
| | 40° C./75% RH | Cracked white to off white powder |
| | 60° C./80% RH | ND |

### Assay Testing

Samples were analyzed by HPLC as previously described and the results for the minor hydrolysis degradation product (Cpd A3) and the major rearrangement product (Cpd A5) are recorded for mass blends in Table 10. For the N-1 design blends, the sum of the hydrolysis and rearrangement products was also recorded in Tables 11 through 15 under the (Total) column.

### Mass Blend Study—Assay and Impurities

TABLE 10
Mass Blend Assay and Impurities

| Mass | | Assay | | Cpd A3 | | Cpd A5 | |
|---|---|---|---|---|---|---|---|
| Blend | Storage | 1M | 3M | 1M | 3M | 1M | 3M |
| 11 | 2-8° C. | 112.5 | 100.0 | ND | ND | 0.1 | ND |
| Excipient | | | | | | | |
| Active | | | | | | | |
| | 25° C./60% RH | 97.1 | 98.7 | ND | ND | 0.1 | ND |
| | 40° C./75% RH | 100.9 | 101.6 | ND | 0.06 | 0.1 | 0.09 |
| | 60° C./80% RH | 98.5 | ND | 0.84 | ND | 0.49 | ND |
| 10 | 2-8° C. | 103.3 | 99.3 | ND | ND | 0.3 | ND |
| Excipient | | | | | | | |
| Active | | | | | | | |
| | 25° C./60% RH | 100.9 | 101.7 | ND | ND | 0.09 | ND |
| | 40° C./75% RH | 101.9 | 100.9 | ND | 0.15 | 0.76 | 1.79 |
| | 60° C./80% RH | 94.3 | ND | 1.39 | ND | 4.85 | ND |

The data surprisingly and unexpectedly showed that the 11 Excipient Active Blend (with DCPD) had a significantly lower level of both degradation products than the 10 Excipient Active Blend without DCPD.

### N-1 Design Study—Assay and Impurities

TABLE 11
14 Day Mean Assay and Impurities (60° C./75% RH)

| Blend 60° C./75% RH 14 Day | Assay | Cpd A3 | Cpd A5 | Total |
|---|---|---|---|---|
| 1 | 98.54 | 0.64 | 0.82 | 1.46 |
| 2 | 98.15 | 0.37 | 1.49 | 1.86 |
| 3 | 98.09 | 0.44 | 1.48 | 1.91 |
| 4 | 98.22 | 0.34 | 1.44 | 1.78 |
| 5 | 98.89 | 0.42 | 0.69 | 1.11 |
| 6 | 98.90 | 0.30 | 0.81 | 1.11 |
| 7 | 99.41 | 0.30 | 0.30 | 0.59 |
| 8 | 99.04 | 0.33 | 0.64 | 0.96 |
| 9 | 99.04 | 0.65 | 0.33 | 0.97 |
| 10 | 98.96 | 0.47 | 0.53 | 1.00 |
| 11 | 98.81 | 0.38 | 0.82 | 1.20 |
| 12 | 98.88 | 0.33 | 0.69 | 1.02 |
| 13 | 99.20 | 0.26 | 0.54 | 0.80 |
| 14 | 98.65 | 0.42 | 0.94 | 1.35 |
| 15 | 98.65 | 0.39 | 0.97 | 1.35 |
| 16 | 98.75 | 0.34 | 0.91 | 1.25 |
| 17 | 99.43 | 0.33 | 0.25 | 0.57 |
| 18 | 99.25 | 0.29 | 0.20 | 0.49 |
| 19 | 99.42 | 0.30 | 0.29 | 0.59 |
| 20 | 99.43 | 0.31 | 0.26 | 0.57 |
| 21 | 99.40 | 0.38 | 0.23 | 0.61 |
| 22 | 99.37 | 0.39 | 0.25 | 0.63 |
| 23 | 99.45 | 0.35 | 0.26 | 0.61 |
| 24 | 99.28 | 0.39 | 0.34 | 0.72 |
| 25 | 99.09 | 0.27 | 0.18 | 0.45 |
| 26 | 99.40 | 0.34 | 0.27 | 0.61 |
| 27 | 99.36 | 0.34 | 0.31 | 0.65 |
| 28 | 99.35 | 0.29 | 0.23 | 0.52 |
| 29 | 99.39 | 0.36 | 0.26 | 0.62 |
| 30 | 99.51 | 0.28 | 0.22 | 0.50 |

TABLE 12
1 Month Mean Assay and Impurities (60° C./75% RH)

| Blend 60° C./75% RH 1 Month | Assay | Cpd A3 | Cpd A5 | Total |
|---|---|---|---|---|
| 1 | 96.99 | 1.87 | 1.15 | 3.02 |
| 2 | 96.21 | 1.21 | 2.58 | 3.79 |
| 3 | 96.52 | 1.30 | 2.19 | 3.48 |
| 4 | 95.96 | 1.07 | 2.97 | 4.03 |
| 5 | 97.91 | 1.25 | 0.85 | 2.09 |
| 6 | 97.66 | 0.95 | 1.08 | 2.03 |
| 7 | 98.50 | 1.06 | 0.45 | 1.51 |
| 8 | 98.10 | 1.03 | 0.87 | 1.90 |
| 9 | 98.04 | 1.65 | 0.31 | 1.96 |
| 10 | 97.80 | 1.49 | 0.71 | 2.20 |
| 11 | 97.77 | 1.14 | 1.10 | 2.23 |
| 12 | 97.75 | 1.06 | 1.20 | 2.26 |
| 13 | 98.18 | 0.87 | 0.94 | 1.81 |
| 14 | 97.61 | 1.23 | 1.17 | 2.40 |
| 15 | 97.54 | 1.18 | 1.28 | 2.46 |
| 16 | 97.69 | 1.04 | 1.28 | 2.32 |
| 17 | 98.60 | 1.10 | 0.30 | 1.40 |
| 18 | 98.27 | 1.00 | 0.20 | 1.20 |
| 19 | 98.66 | 1.04 | 0.30 | 1.34 |
| 20 | 98.72 | 0.99 | 0.29 | 1.27 |
| 21 | 98.58 | 1.23 | 0.20 | 1.43 |
| 22 | 98.51 | 1.26 | 0.24 | 1.50 |
| 23 | 98.64 | 1.10 | 0.27 | 1.37 |
| 24 | 98.36 | 1.21 | 0.44 | 1.65 |
| 25 | 98.04 | 0.90 | 0.24 | 1.14 |
| 26 | 98.57 | 1.12 | 0.31 | 1.43 |
| 27 | 98.55 | 1.13 | 0.33 | 1.46 |
| 28 | 98.54 | 0.91 | 0.26 | 1.17 |
| 29 | 98.41 | 1.17 | 0.43 | 1.60 |
| 30 | 99.10 | 0.68 | 0.23 | 0.91 |

TABLE 13
1 Month Mean Assay and Impurities (40° C./75% RH)

| Blend 40° C./75% RH 1 Month | Assay | Cpd A3 | Cpd A5 | Total |
|---|---|---|---|---|
| 1 | 99.82 | 0.05 | 0.14 | 0.19 |
| 2 | 99.67 | 0.04 | 0.30 | 0.34 |
| 3 | 99.45 | 0.06 | 0.50 | 0.56 |
| 4 | 98.43 | 0.06 | 1.17 | 1.23 |
| 5 | 99.73 | 0.05 | 0.23 | 0.28 |
| 6 | 99.31 | 0.05 | 0.65 | 0.70 |
| 7 | 99.89 | 0.04 | 0.08 | 0.12 |
| 8 | 99.75 | 0.04 | 0.22 | 0.26 |
| 9 | 99.87 | 0.05 | 0.08 | 0.13 |
| 10 | 99.86 | 0.04 | 0.10 | 0.14 |
| 11 | 99.76 | 0.04 | 0.21 | 0.25 |
| 12 | 99.74 | 0.03 | 0.24 | 0.27 |
| 13 | 98.62 | 0.06 | 1.32 | 1.38 |
| 14 | 99.48 | 0.06 | 0.47 | 0.52 |
| 15 | 99.14 | 0.06 | 0.81 | 0.87 |
| 16 | 98.09 | 0.07 | 1.84 | 1.91 |
| 17 | 99.88 | 0.04 | 0.09 | 0.13 |
| 18 | 99.77 | 0.04 | 0.19 | 0.23 |
| 19 | 99.83 | 0.04 | 0.15 | 0.18 |
| 20 | 99.78 | 0.04 | 0.19 | 0.23 |
| 21 | 99.80 | 0.05 | 0.16 | 0.20 |
| 22 | 99.77 | 0.04 | 0.19 | 0.23 |
| 23 | 99.79 | 0.04 | 0.17 | 0.21 |
| 24 | 99.76 | 0.04 | 0.21 | 0.25 |
| 25 | 99.42 | 0.04 | 0.54 | 0.58 |
| 26 | 99.89 | 0.04 | 0.09 | 0.13 |
| 27 | 99.88 | 0.04 | 0.09 | 0.13 |
| 28 | 99.84 | 0.03 | 0.13 | 0.16 |
| 29 | 99.68 | 0.05 | 0.06 | 0.10 |
| 30 | 99.91 | 0.03 | 0.06 | 0.09 |

TABLE 14
2 Month Mean Assay and Impurities (40° C./75% RH)

| Blend 40° C./75% RH 2 Month | Assay | Cpd A3 | Cpd A5 | Total |
|---|---|---|---|---|
| 1 | 99.61 | 0.10 | 0.29 | 0.39 |
| 2 | 99.44 | 0.09 | 0.48 | 0.57 |
| 3 | 99.26 | 0.11 | 0.64 | 0.75 |
| 4 | 98.46 | 0.11 | 1.43 | 1.54 |
| 5 | 99.66 | 0.10 | 0.25 | 0.35 |
| 6 | 99.11 | 0.10 | 0.80 | 0.90 |
| 7 | 99.80 | 0.08 | 0.12 | 0.20 |
| 8 | 99.61 | 0.08 | 0.31 | 0.39 |
| 9 | 99.78 | 0.11 | 0.11 | 0.22 |
| 10 | 99.78 | 0.09 | 0.13 | 0.22 |
| 11 | 99.56 | 0.09 | 0.36 | 0.45 |
| 12 | 99.61 | 0.08 | 0.32 | 0.39 |
| 13 | 98.28 | 0.11 | 1.61 | 1.72 |
| 14 | 99.18 | 0.11 | 0.72 | 0.82 |
| 15 | 99.15 | 0.11 | 0.75 | 0.86 |
| 16 | 97.99 | 0.12 | 1.90 | 2.02 |
| 17 | 99.78 | 0.08 | 0.14 | 0.22 |
| 18 | 99.72 | 0.08 | 0.20 | 0.28 |
| 19 | 99.83 | 0.07 | 0.10 | 0.17 |
| 20 | 99.77 | 0.08 | 0.16 | 0.24 |
| 21 | 99.78 | 0.10 | 0.13 | 0.23 |
| 22 | 99.76 | 0.09 | 0.15 | 0.24 |
| 23 | 99.75 | 0.09 | 0.17 | 0.26 |
| 24 | 99.69 | 0.10 | 0.22 | 0.32 |
| 25 | 99.35 | 0.09 | 0.56 | 0.65 |
| 26 | 99.77 | 0.09 | 0.15 | 0.24 |
| 27 | 99.66 | 0.09 | 0.27 | 0.35 |
| 28 | 99.75 | 0.08 | 0.18 | 0.26 |
| 29 | 99.73 | 0.10 | 0.17 | 0.27 |
| 30 | 99.72 | 0.07 | 0.22 | 0.29 |

TABLE 15
3 Month Mean Assay and Impurities (40° C./75% RH)

| Blend 40° C./75% RH 3 Month | Assay | Cpd A3 | Cpd A5 | Total |
|---|---|---|---|---|
| 1 | 99.54 | 0.16 | 0.31 | 0.46 |
| 2 | 99.32 | 0.14 | 0.54 | 0.68 |
| 3 | 99.10 | 0.18 | 0.73 | 0.90 |
| 4 | 98.22 | 0.18 | 1.61 | 1.79 |
| 5 | 99.48 | 0.16 | 0.36 | 0.52 |
| 6 | 98.23 | 0.16 | 0.89 | 1.05 |
| 7 | 99.70 | 0.13 | 0.18 | 0.31 |
| 8 | 99.44 | 0.13 | 0.44 | 0.57 |
| 9 | 99.66 | 0.17 | 0.18 | 0.35 |
| 10 | 99.62 | 0.15 | 0.24 | 0.39 |
| 11 | 99.45 | 0.15 | 0.40 | 0.55 |
| 12 | 99.42 | 0.14 | 0.44 | 0.58 |
| 13 | 97.98 | 0.17 | 1.86 | 2.03 |
| 14 | 99.15 | 0.17 | 0.69 | 0.86 |
| 15 | 98.81 | 0.17 | 1.04 | 1.21 |
| 16 | 97.88 | 0.18 | 1.95 | 2.13 |
| 17 | 99.44 | 0.15 | 0.41 | 0.56 |
| 18 | 99.64 | 0.14 | 0.22 | 0.36 |
| 19 | 99.75 | 0.12 | 0.13 | 0.25 |
| 20 | 99.70 | 0.14 | 0.18 | 0.31 |
| 21 | 99.71 | 0.15 | 0.15 | 0.30 |
| 22 | 99.69 | 0.15 | 0.17 | 0.32 |
| 23 | 99.65 | 0.15 | 0.21 | 0.35 |
| 24 | 99.65 | 0.16 | 0.19 | 0.35 |
| 25 | 99.25 | 0.14 | 0.61 | 0.75 |
| 26 | 99.69 | 0.14 | 0.17 | 0.31 |
| 27 | 99.73 | 0.14 | 0.14 | 0.28 |
| 28 | 99.66 | 0.14 | 0.21 | 0.34 |
| 29 | 99.64 | 0.17 | 0.20 | 0.37 |
| 30 | 99.65 | 0.12 | 0.23 | 0.35 |

### N-1 Design Study—Excipient Comparison

The following tables show the mean total degradation products for blends stored at under the indicated stability conditions. The Mean Degradant Total is the sum of the percentage of the Compound A3 hydrolysis product and the sum of the percentage of the Compound A5 rearrangement product for blends that contain or do not contain the excipient.

TABLE 16
1 Month Mean Degradant Total (60° C./75% RH)

| | Mean Degradant Total | Mean Degradant |
|---|---|---|
| Ingredient | without Ingredient | Total with Ingredient |
| F1 | 1.85% | 2.1% |
| F2 | 2.35% | 1.35% |
| D1 | 1.94% | 1.96% |
| D2 | 1.8% | 2.15% |
| W | 1.55% | 2.15% |
| B1 | 1.9% | 2.0% |
| B2 | 1.9% | 2.0% |
| Fa | 1.7% | 2.05% |

For Table 16, comparing Mean Degradant Total with and without the ingredient, the results show that for F1 and F2 (the 2 fillers), the mean degradant level for all blends containing F1 was approximately 2.1% and the level for F2 was approximately 1.4%. Therefore, this data would indicate that one skilled in the art would choose F2 over F1 for the formulation of the present invention. Similarly, comparing disintegrants, D1 would be chosen over D2 for the instant formulation.

TABLE 17
1 Month Mean Degradant Total (60° C./75% RH)

| | Mean Degradant Total | Mean Degradant |
|---|---|---|
| Ingredient | without Ingredient | Total with Ingredient |
| F1 | 98.07% | 97.88% |
| F2 | 97.65% | 98.50% |
| D1 | 97.96% | 98.05% |
| D2 | 98.15% | 97.76% |
| W | 98.39% | 97.79% |
| B1 | 98.01% | 97.98% |
| B2 | 98.11% | 97.80% |
| Fa | 98.25% | 97.86% |

For Table 17, comparing Mean Degradant Total with and without the ingredient, the results show that for the 2 fillers, the mean degradant level for all blends containing F2 was slightly higher than the level for blends containing F1. Comparing disintegrants, the level for D2 was slightly lower than the level for D1. The scale for the data obtained from the lower level to the higher level ranges from 97.65% to 98.50%.

TABLE 18
3 Month Mean Degradant Total (40° C./75% RH)

| | Mean Degradant Total | Mean Degradant |
|---|---|---|
| Ingredient | without Ingredient | Total with Ingredient |
| F1 | 0.50% | 0.88% |
| F2 | 0.84% | 0.37% |
| D1 | 0.82% | 0.40% |
| D2 | 0.45% | 0.96% |
| W | 0.59% | 0.68% |
| B1 | 0.75% | 0.51% |
| B2 | 0.48% | 0.92% |
| Fa | 0.63% | 0.66% |
| A Base | 0.35% | 0.66% |

For Table 18, compared to Table 16, the Mean Degradant Total for each blend with and without the ingredient is similar. Blends with F2 had a lower degradant level than blends with F1. Blends with D1 had a lower degradant level than blends with D2. Blends with B1 had a lower degradant level than blends with B2. Blends with W had a slightly higher degradant level than blends without W. Blends with Fa showed practically no difference. The degradant level with the base blend (A Base) is compared to the level with pure API alone.

TABLE 19
3 Month Mean Degradant Total (60° C./75% RH)

| | Mean Degradant Total | Mean Degradant |
|---|---|---|
| Ingredient | without Ingredient | Total with Ingredient |
| F1 | 99.49% | 99.06% |
| F2 | 99.12% | 99.62% |
| D1 | 99.14% | 99.60% |
| D2 | 99.51% | 99.04% |
| W | 99.41% | 99.27% |
| B1 | 99.22% | 99.48% |
| B2 | 99.52% | 99.03% |
| Fa | 99.37% | 99.30% |
| A Base | 99.64% | 99.31% |

For Table 19, compared to Table 17, the Mean Degradant Total for each blend with and without the ingredient was similar. Blends with F2 had a slightly higher degradant level than the level for blends containing F1. The level for D1 was slightly higher than the level for D1. Blends without W had a slightly higher degradant level than blends with W. Blends with B2 had a slightly lower degradant level than blends with B1. Blends with Fa showed practically no difference. The degradant level with the base blend (A Base) is compared to the level with pure API alone. The scale for the data obtained from the lower level to the higher level ranges from 99.03% to 99.64%.

It is to be understood that the preceding description of the invention and various examples thereof have emphasized certain aspects. Numerous other equivalents not specifically elaborated on or discussed may nevertheless fall within the spirit and scope of the present invention or the following claims and are intended to be included.

What is claimed is:

1. A composition of a phenylalkyl carbamate compound comprising an admixture of the compound with an effective amount of one or more excipients wherein at least one excipient is dibasic calcium phosphate dihydrate, whereby the dibasic calcium phosphate dihydrate reduces degradation of the phenylalkyl carbamate compound in the composition.

2. The composition of claim 1, wherein the compound is a compound of formula (I): 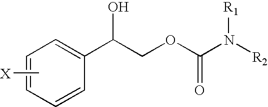
or a form thereof wherein
phenyl is substituted at X with one to five halogen atoms independently selected from the group consisting of fluorine, chlorine, bromine and iodine; and,
R₁ and R₂ are independently selected from the group consisting of hydrogen and C₁₋₄alkyl; wherein C₁₋₄alkyl is optionally substituted with phenyl, wherein phenyl is optionally substituted with substituents independently selected from the group consisting of halogen, C₁₋₄alkyl, C₁₋₄alkoxy, amino, nitro and cyano.

3. The composition of claim 2, wherein said compound is carbamic acid 2-(2-chloro-phenyl)-2-hydroxy-ethyl ester.

4. The composition of claim 2, wherein said compound is carbamic acid (2R)-2-(2-chloro-phenyl )-2-hyd roxy-ethyl ester.

5. The composition of claim 4, wherein said compound predominates in a range of from about 75% or greater; or in a range of from about 90% or greater; or in a range of from about 95% or greater; or in a range of from about 98% or greater; or in a range of from about 99% or greater.

6. The composition of claim 2, wherein said compound is carbamic acid (2S)-2-(2-chloro-phenyl )-2-hydroxy-ethyl ester.

7. The composition of claim 6, wherein said compound predominates in a range of from about 75% or greater; or in a range of from about 90% or greater; or in a range of from about 95% or greater; or in a range of from about 98% or greater; or in a range of from about 99% or greater.

8. The composition of claim 1, wherein said dibasic calcium phosphate dihydrate is unmilled.

9. The composition of claim 8, wherein said dibasic calcium phosphate dihydrate has a pH in a range of from about 5.0 to a pH of about 5.8; or a pH in a range of from about 5.1 to a pH of about 5.7; or a pH in a range of from about 5.2 to a pH of about 5.6; or a pH in a range of from about 5.3 to a pH of about 5.5; or a pH in a range of about 5.4.

10. The composition of claim 1, wherein said effective amount of dibasic calcium phosphate dihydrate is in a range of from about 4% (w/w) to about 40% (w/w).

11. The composition of claim 1, wherein said effective amount of dibasic calcium phosphate dihydrate is in a range of from about 4% (w/w) to about 35% (w/w).

12. The composition of claim 1, wherein said effective amount of dibasic calcium phosphate dihydrate is in a range of from about 4% (w/w) to about 30% (w/w).

13. The composition of claim 1, wherein said effective amount of dibasic calcium phosphate dihydrate is in a range of from about 4% (w/w) to about 25% (w/w).

14. The composition of claim 1, wherein said effective amount of dibasic calcium phosphate dihydrate is in a range of from about 4% (w/w) to about 20% (w/w).

15. The composition of claim 1, wherein said effective amount of dibasic calcium phosphate dihydrate is in a range of from about 4% (w/w) to about 10% (w/w).

16. The composition of claim 1, wherein said effective amount of dibasic calcium phosphate dihydrate is about 4% (w/w).

17. The composition of claim 1, wherein the composition remains stable for a period of time in a range of about 6 months to about 5 years; or, in a range of from about one year to about 5 years; or, in a range of from about 2 years to about 5 years; or, in a range of from about 3 years to about 5 years; or, in a range of from about 4 years to about 5 years; or, in a range of about 5 years, when stored under ambient conditions.

18. The composition of claim 1, wherein one or more excipients are selected from microcrystalline cellulose, hydroxypropyl methylcellulose, lactose, mannitol, sodium starch glycolate, cross-linked polyplasdone, polyethylene glycol, sodium lauryl sulfate, magnesium stearate, sodium stearyl fumarate or colloidal silicon dioxide.

19. The composition of claim 18, wherein one or more excipients are selected from hydroxypropyl methylcellulose, sodium starch glycolate, cross-linked polyplasdone, polyethylene glycol, sodium lauryl sulfate or colloidal silicon dioxide.

20. The composition of claim 1, wherein one or more excipients are selected from hydroxypropyl methylcellulose or sodium starch glycolate.

21. The composition of claim 1, wherein said composition is a tablet.

22. The composition of claim 21, wherein one or more excipients are selected from microcrystalline cellulose, hydroxypropyl methylcellulose, lactose, mannitol, sodium starch glycolate, cross-linked polyplasdone, polyethylene glycol, sodium lauryl sulfate, magnesium stearate, sodium stearyl fumarate or colloidal silicon dioxide.

23. The composition of claim 21, wherein one or more excipients are selected from hydroxypropyl methylcellulose, sodium starch glycolate, cross-linked polyplasdone, polyethylene glycol, sodium lauryl sulfate or colloidal silicon dioxide.

24. The composition of claim 21, wherein one or more excipients are selected from hydroxypropyl methylcellulose or sodium starch glycolate.

25. The composition of claim 21, wherein the compound is the compound of claim 3.

26. The composition of claim 21, wherein the compound is the compound of claim 4.

27. The composition of claim 26, wherein said compound predominates in a range of from about 75% or greater; or in a range of from about 90% or greater; or in a range of from about 95% or greater; or in a range of from about 98% or greater; or in a range of from about 99% or greater.

28. The composition of claim 1, further comprising one or more therapeutic agents.

29. A method of preparing a composition comprising the step of admixing an effective amount of one or more excipients wherein at least one excipient is dibasic calcium phosphate dihydrate with a compound of formula (I): 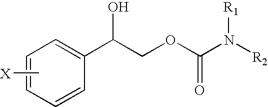
or a form thereof wherein
phenyl is substituted at X with one to five halogen atoms independently selected from the group consisting of fluorine, chlorine, bromine and iodine; and,
R₁ and R₂ are independently selected from the group consisting of hydrogen and C₁₋₄alkyl; wherein C₁₋₄alkyl is optionally substituted with phenyl, wherein phenyl is optionally substituted with substituents independently selected from the group consisting of halogen, C₁₋₄alkyl, C₁₋₄alkoxy, amino, nitro and cyano.

30. The method of claim 29, wherein the compound is the compound of claim 3.

31. The method of claim 29, wherein the compound is the compound of claim 4.

32. The method of claim 31, wherein said compound predominates in a range of from about 75% or greater; or in a range of from about 90% or greater; or in a range of from about 95% or greater; or in a range of from about 98% or greater; or in a range of from about 99% or greater.

33. A method for treatment of a CNS disorder in a subject in need thereof comprising the step of administering to the subject an effective amount of a composition comprising an effective amount of one or more excipients wherein at least one excipient is dibasic calcium phosphate dihydrate and a compound of formula (I): 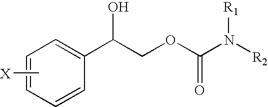
or a form thereof wherein
phenyl is substituted at X with one to five halogen atoms independently selected from the group consisting of fluorine, chlorine, bromine and iodine; and,
R₁ and R₂ are independently selected from the group consisting of hydrogen and C₁₋₄alkyl; wherein C₁₋₄alkyl is optionally substituted with phenyl, wherein phenyl is optionally substituted with substituents independently selected from the group consisting of halogen, C₁₋₄alkyl, C₁₋₄alkoxy, amino, nitro and cyano.

34. The method of claim 33, wherein the compound is the compound of claim 3.

35. The method of claim 33, wherein the compound is the compound of claim 4.

36. The method of claim 35, wherein said compound predominates in a range of from about 75% or greater; or in a range of from about 90% or greater; or in a range of from about 95% or greater; or in a range of from about 98% or greater; or in a range of from about 99% or greater.

37. The method of claim 33, wherein the CNS disorder is selected from convulsions, epilepsy, stroke and muscle spasm; useful in the treatment of central nervous system diseases, particularly as anticonvulsants, antiepileptics, neuroprotective agents and centrally acting muscle relaxants; useful in treating and preventing neuropathic pain, cluster and migraine headache pain, bipolar disorder, chronic and acute neurodegenerative disorders, psychotic disorders, movement disorders, addictive disorders, impulse control disorders, anxiety disorders, antiepileptogenesis and for the treatment of pain.

38. A composition resulting from a method of preparation comprising the step of admixing an effective amount of one or more excipients wherein at least one excipient is dibasic calcium phosphate dihydrate with a compound of formula (I): 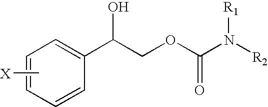
or a form thereof wherein
phenyl is substituted at X with one to five halogen atoms independently selected from the group consisting of fluorine, chlorine, bromine and iodine; and,
R₁ and R₂ are independently selected from the group consisting of hydrogen and C₁₋₄alkyl; wherein C₁₋₄alkyl is optionally substituted with phenyl, wherein phenyl is optionally substituted with substituents independently selected from the group consisting of halogen, C₁₋₄alkyl, C₁₋₄alkoxy, amino, nitro and cyano.

39. The composition of claim 38, wherein the compound is the compound of claim 3.

40. The composition of claim 38, wherein the compound is the compound of claim 4.

41. The composition of claim 40, wherein said compound predominates in a range of from about 75% or greater; or in a range of from about 90% or greater; or in a range of from about 95% or greater; or in a range of from about 98% or greater; or in a range of from about 99% or greater.

42. A tablet comprising an effective amount of dibasic calcium phosphate dihydrate and one or more excipients selected from microcrystalline cellulose, hydroxypropyl methylcellulose, lactose, mannitol, sodium starch glycolate, cross-linked polyplasdone, polyethylene glycol, sodium lauryl sulfate, magnesium stearate, sodium stearyl fumarate or colloidal silicon dioxide and a compound of formula (I): 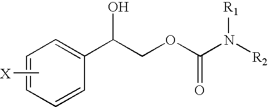
or a form thereof wherein
phenyl is substituted at X with one to five halogen atoms independently selected from the group consisting of fluorine, chlorine, bromine and iodine; and,
R₁ and R₂ are independently selected from the group consisting of hydrogen and C₁₋₄alkyl; wherein C₁₋₄alkyl is optionally substituted with phenyl, wherein phenyl is optionally substituted with substituents independently selected from the group consisting of halogen, C₁₋₄alkyl, C₁₋₄alkoxy, amino, nitro and cyano.

43. The tablet of claim 42, wherein the compound is the compound of claim 3.

44. The tablet of claim 42, wherein the compound is the compound of claim 4.

45. The tablet of claim 44, wherein said compound predominates in a range of from about 75% or greater; or in a range of from about 90% or greater; or in a range of from about 95% or greater; or in a range of from about 98% or greater; or in a range of from about 99% or greater.

46. The tablet of claim 42, wherein one or more excipients are selected from hydroxypropyl methylcellulose, sodium starch glycolate, cross-linked polyplasdone, polyethylene glycol, sodium lauryl sulfate or colloidal silicon dioxide.

47. The tablet of claim 42, wherein one or more excipients are selected from hydroxypropyl methylcellulose or sodium starch glycolate.

48. The tablet of claim 42, wherein said effective amount of dibasic calcium phosphate dihydrate is in a range of from about 4% (w/w) to about 40% (w/w).

49. The tablet of claim 42, wherein said effective amount of dibasic calcium phosphate dihydrate is in a range of from about 4% (w/w) to about 35% (w/w).

50. The tablet of claim 42, wherein said effective amount of dibasic calcium phosphate dihydrate is in a range of from about 4% (w/w) to about 30% (w/w).

51. The tablet of claim 42, wherein said effective amount of dibasic calcium phosphate dihydrate is in a range of from about 4% (w/w) to about 25% (w/w).

52. The tablet of claim 42, wherein said effective amount of dibasic calcium phosphate dihydrate is in a range of from about 4% (w/w) to about 20% (w/w).

53. The tablet of claim 42, wherein said effective amount of dibasic calcium phosphate dihydrate is in a range of from about 4% (w/w) to about 10% (w/w).

54. The tablet of claim 42, wherein said effective amount of dibasic calcium phosphate dihydrate is about 4% (w/w).

55. Use of the composition of claim 1 in the manufacture of a medicament for the treatment of CNS disorders.

56. The use of claim 55, wherein the CNS disorder is selected from convulsions, epilepsy, stroke and muscle spasm; useful in the treatment of central nervous system diseases, particularly as anticonvulsants, antiepileptics, neuroprotective agents and centrally acting muscle relaxants; useful in treating and preventing neuropathic pain, cluster and migraine headache pain, bipolar disorder, chronic and acute neurodegenerative disorders, psychotic disorders, movement disorders, addictive disorders, impulse control disorders, anxiety disorders, antiepileptogenesis and for the treatment of pain.
